(19) United States
(12) Patent Application Publication
(10) Pub. No.: US 2022/0039873 A1
(43) Pub. Date: Feb. 10, 2022

(54) ULTRASOUND GUIDANCE SYSTEM AND METHOD

(71) Applicant: Melvyn Harris, Folsom, CA (US)

(72) Inventor: Melvyn Harris, Folsom, CA (US)

(21) Appl. No.: 17/393,476

(22) Filed: Aug. 4, 2021

Related U.S. Application Data
(60) Provisional application No. 63/062,250, filed on Aug. 6, 2020.

(51) Int. Cl. A61B 34/20; G06T 7/70; G06T 11/00; A61B 90/00; A61B 8/00; A61B 8/08; G02B 27/01

(52) U.S. Cl. CPC A61B 34/20; G06T 7/70; G06T 11/00; A61B 90/37; A61B 8/4263; A61B 8/5207; G02B 2027/014; A61B 8/5261; G02B 27/0101; G06T 2207/10132; A61B 2034/2055; A61B 2090/365; G02B 2027/0138; A61B 8/08

(57) ABSTRACT

An ultrasound guidance system and method include an augmented reality device, a camera, and a computing system. The computing system receives a user input from a user including a target internal anatomical structure of the patient. The computing system then processes image data of surface anatomy of the patient captured by the camera. The computing system processes the image data to identify surface anatomical structures to locate underlying internal anatomy of the patient. The computing system uses this information to determine a probe location on a surface of the patient in which an ultrasound probe is capable of imaging the target internal anatomical structure of the patient when placed against the surface. The computing system then generates output data for a display of the augmented reality device including a target indicia that overlays an image of the patient on the display at the probe location.

This application claims benefit of priority of U.S. provisional application No. 63/062,250, filed Aug. 6, 2020, the contents of which are herein incorporated by reference.

### FIELD

The present invention relates to medical ultrasound and methods of performing medical ultrasound.

### BACKGROUND

Medical ultrasound includes diagnostic imaging techniques, as well as therapeutic applications of ultrasound. In diagnosis, it is used to create an image of internal body structures such as tendons, muscles, joints, blood vessels, and internal organs. Its aim is usually to find a source of disease, to exclude pathology, or to assist in medical procedures. The practice of examining pregnant women using ultrasound is called obstetric ultrasound, and was an early development of clinical ultrasonography.

Ultrasound is composed of sound waves with frequencies which are significantly higher than the range of human hearing (>20,000 Hz). Ultrasonic images, also known as sonograms, are created by sending pulses of ultrasound into tissue using a probe. The ultrasound pulses echo off tissues with different reflection properties and are returned to the probe which records and displays them as an image.

Compared to other medical imaging modalities, ultrasound has several advantages. It is noninvasive, provides images in real-time, is portable, and can consequently be brought to the bedside. It is substantially lower in cost than other imaging strategies and does not use harmful ionizing radiation. In recent years ultrasound has become more widely available as the size of the technology has become smaller and the technology is much less expensive than before.

Sonography (ultrasonography) is widely used in medicine. It is possible to perform both diagnosis and therapeutic procedures, using ultrasound to guide interventional procedures such as biopsies or to drain collections of fluid, which can be both diagnostic and therapeutic. Sonographers are medical technicians who perform scans which are traditionally interpreted by radiologists, physicians who specialize in the application and interpretation of medical imaging modalities. Increasingly, non-radiologist physicians and other medical practitioners, like nurses and medics who provide direct patient care are using ultrasound in office, hospital and pre-hospital practice.

Medical practitioners must undergo extensive training to become a certified sonographer. The medical practitioners must learn where to place the ultrasound probe on the body to obtain ultrasound images of target organs or structures for either diagnostic or procedural purposes. The training is lengthy and expensive. Thus, capable medical practitioners are deterred from certifying and using ultrasound.

Accordingly, it would be desirable for medical practitioners untrained in sonography to be able to effectively capture sonographs.

### SUMMARY

The present invention relates to an ultrasound guidance system and method.

A feature of the present invention is to allow medical practitioners to capture ultrasound images without formal training in sonography.

A further feature of the present invention is to provide a system and method of guiding medical practitioners to capture accurate ultrasound images.

A further feature of the present invention is to notify medical practitioners when an ultrasound probe is placed at a target location of a patient.

Additional features and advantages of the present invention will be set forth in part in the description that follows, and in part will be apparent from the description, or may be learned by practice of the present invention. The objectives and other advantages of the present invention will be realized and attained by means of the elements and combinations particularly pointed out in the description and appended claims.

To achieve these and other advantages, and in accordance with the purposes of the present invention, as embodied and broadly described herein, the present invention, in part, relates to a guidance system. The guidance system includes an augmented reality device having a display, a camera configured to capture image data, and a computing system. The computing system includes a processor and a memory. The memory stores computer-readable instructions that, upon execution by the processor, configure the computing system to perform steps. The computing system receives a user input from a user, the user input including a target internal anatomical structure of the patient. The computing system processes the image data captured by the camera, the image data including at least an image of surface anatomy of the patient. Processing the image data includes identifying at least one surface anatomical structure of the patient to locate internal anatomy of the patient. The computing system determines a probe location based on the processed image data and the user input. The probe location is on a surface of the patient where an ultrasound probe is capable of imaging the target internal anatomical structure of the patient when placed against the surface. The computing system generates output data for the display of the augmented reality device. The output data includes a target indicia. The target indicia overlays an image of the patient on the display at the probe location.

The present invention further relates to a method of guiding a user to capture ultrasound images. The method includes the following steps: entering a user input to a computing system, the user input including a target internal anatomical structure of a patient; capturing, with a camera, image data of surface anatomy of the patient; processing, with the computing system, the image data captured by the camera, wherein processing the image data includes identifying at least one surface anatomical structure of the patient to locate internal anatomy of the patient; determining, with the computing system, a probe location on a surface of the patient where an ultrasound probe is capable of imaging the target internal anatomical structure of the patient when placed against the surface, based on the processed image data and the user input; and generating, with the computing system, output data for the display of the augmented reality device, the output data including a target indicia, wherein the target indicia overlays an image of the patient on the display at the probe location.

It is to be understood that both the foregoing general description and the following detailed description are exemplary and explanatory only and are intended to provide a further explanation of the present invention, as claimed.

### BRIEF DESCRIPTION OF THE DRAWINGS

FIG. 1 is a schematic view of an embodiment of the present invention, illustrating components of an ultrasound guidance system.

FIG. 2 is a front view of a computing system of an embodiment of the present invention, illustrating a plurality of ultrasound targets for a user to select.

FIG. 3 is a schematic view of an embodiment of the present invention, illustrating a user capturing image data of a patient.

FIG. 4A is a view of an exemplary image of a patient captured by a camera of an embodiment of the present invention.

FIG. 4B is a view of the image of FIG. 4A, the image showing reference lines that represent spatial relationships between surface anatomical structures utilized during data processing.

FIG. 4C is a view of the image of FIG. 4A, the image showing reference lines and markers that represent spatial relationships between surface anatomical structures utilized during data processing.

FIG. 4D is a view of an image of a side of a patient, the image showing reference lines utilized during data processing.

FIG. 5 is a schematic view of an embodiment of the present invention, illustrating a target indicia overlayed on an image of a patient.

FIG. 6 is a schematic view of an embodiment of the present invention, illustrating a target indicia overlayed on an image of a patient.

### DETAILED DESCRIPTION

An ultrasound guidance system and method are described herein. According to the present invention, the ultrasound guidance system and method utilizes an augmented reality device that can precisely display to a medical user a target region to place an ultrasound probe and achieve a desired ultrasound image with minimal training and expense, while standardizing the procedure between users. The present invention greatly reduces the need for training and allows a greater number of medical practitioners, such as medics and nurses, to obtain useful ultrasound images of a patient.

References herein to “an example” or “a specific example” or “an aspect” or “an embodiment,” or similar phrases, are intended to introduce a feature or features of the apparel, or components thereof, or methods of using or manufacturing the apparel (depending on context), and that can be combined with any combination of previously-described or subsequently-described examples, aspects, embodiments (i.e. features), unless a particular combination of features is mutually exclusive or if context indicates otherwise. Further, as used in this specification, the singular forms “a,” “an,” and “the” include plural referents (e.g. at least one or more) unless the context clearly dictates otherwise.

The present invention includes a guidance system. The guidance system includes at least an augmented reality device having a display, a camera configured to capture image data, and a computing system. The computing system includes at least a processor and a memory. The memory stores computer-readable instructions that, upon execution by the processor, configure the computing system to perform steps. The computing system receives a user input from a user, the user input including a target internal anatomical structure of the patient. The computing system processes the image data captured by the camera, the image data including at least an image of surface anatomy of the patient. Processing the image data includes identifying at least one surface anatomical structure of the patient to locate internal anatomy of the patient. The computing system determines a probe location based on the processed image data and the user input. The probe location is on a surface of the patient in which an ultrasound probe is capable of imaging the target internal anatomical structure of the patient when placed against the surface. The computing system generates output data for the display of the augmented reality device. The output data includes a target indicia. The target indicia overlays an image of the patient on the display at the probe location.

The augmented reality device can include the computing system, the display, and the camera. Alternatively, the augmented reality device can be separate from the computing system and the camera. In such embodiments, the computing system, the augmented reality device, and the camera can communicate with one another via a hard-wired interface, a wireless interface, or a combination thereof.

The augmented reality device can include a smart device. The smart device can be, for example, a smart phone, a tablet, a smart watch, smart glasses, or the like. The smart device can be a smart phone that can include, for example, a touchscreen interface. The computer-readable instructions can be in the form of application software loaded on the memory, for example, an app loaded on a smart phone or tablet.

The smart device can be in the form of a head mount, such as smart glasses. The head mount can include the computing system, the display, the camera, and other sensors. The display of the head mount can be at least partially transparent. The at least partially transparent display is configured to display augmentation graphics such as semi-opaque images that appear to a user to be superimposed on at least a portion of a natural field of view of the user.

The guidance system of the present invention can further include an ultrasound probe. The ultrasound probe can be configured to communicate with the computing system over a hard-wired interface or a wireless interface. The ultrasound probe can house internal and external components. Internally, the ultrasound probe can utilize a piezoelectric ultrasound transducer, semiconductor chip, or a combination thereof. The ultrasound probe can further include an analog to digital signal converter, a wireless transmitter, and battery. A button can be on the exterior of the ultrasound encasement that allows for functional interaction with the software and device operability. Externally, the ultrasound probe can include a USB port for charging and a screen that displays Wi-Fi connectivity, battery level, and/or other data regarding the ultrasound probe.

As mentioned above, the memory of the computing system stores computer-readable instructions that, upon execution by the processor, configure the computing system to perform steps. The computing system first receives a user input from a user. The user input includes a target internal anatomical structure of the patient that is intended to be imaged by ultrasound. The user of the present invention can start the process by selecting a target internal anatomical structure either on the computing system, the ultrasonic probe, the augmented reality device, or a combination thereof. Selection can be done by a user interface, such as but not limited to a touchscreen interface, a voice command interface, a motion command interface, a keyboard, a mouse, by other user interfaces, or combinations thereof. In certain embodiments, the user selects a target internal anatomical structure from a display menu that provides a plurality of target internal anatomical structures to choose from. For example, the user can select the liver, the gall bladder, the kidneys, the lungs, the heart, the stomach, or the like from a list of target internal anatomical structures. Once selected, the computing system can store the user input on the memory.

The target internal anatomical structure can also include an ultrasound imaging procedure. An ultrasound imaging procedure includes targets of multiple internal anatomical structures. If the user selects an ultrasound imaging procedure, the computing system guides the user to image each of the different internal anatomical structures. Examples of ultrasound imaging procedures that a user can choose from can include focused abdominal sonography for trauma (FAST exam), rapid ultrasound for shock and hypotension (RUSH exam), peripheral intravenous access, radial artery blood gas sampling, radial artery arterial line placement, internal or femoral vein catheter insertion, or the like.

Once the user selects a target internal anatomical structure, the user can point the camera towards the patient. The patient may or may not be clothed depending on the target internal anatomical structure. For example, if the target internal anatomical structure is in the neck, the patient can be clothed as long as the neck of the patient is exposed. If the target internal anatomical structure is in the abdomen, the patient can remove their shirt such that the computing system identifies the surface anatomical structures of the abdomen in order to determine the probe location on the surface of the patient. The user can direct the camera to acquire image data of the patient. The image data can be in the form of a still image, a video, or a live feed of the patient. For example, the user can prompt the camera to capture an image or video of the patient. Alternatively, the user can simply point the camera in the patient's direction while the camera is turned on and the computing system can recognize a live feed of the patient.

In certain embodiments, the computing system can direct the user where to capture an image or images of the patient. For example, if the target internal anatomical structure, the computing system can provide directions for what images of the patient are needed. The computing system can display on the display or communicate via the user interface that the patient needs to remove their shirt and the user needs to capture a front of the chest of the patient. If the computing system needs more than one image of the patient to properly determine a probe location, the computing system can direct the user to capture multiple images of different areas of the patient view the display and/or the user interface, such as the front and the side, the front and the back, the inside and outside of the arm, and the like.

Once the images are captured, the computing system recognizes the image data of the patient and then processes the image data. The computing system processes the image data by identifying surface anatomical structures of the patient to locate internal anatomy of the patient. In certain embodiments, the computing system recognizes one or more particular surface anatomical structures of the patient. For example, the camera can be an infrared camera that uses infrared laser scatter beam technology to recognize surface anatomical structures of the patient. In particular, the computing system can use the infrared camera to create a three-dimensional reference map of the face and body of the patient and compare the three-dimensional reference map to reference data stored in the memory of the computing system. In certain embodiments, the computing system can identify the surface anatomical structures based on user input, such as touch, hand gesture, voice activation or the like, from the user.

In certain embodiments, the computing system identifies the surface anatomical structures of the patient by using machine learning or artificial intelligence algorithms to identify particular parts of the body of the patient. The computing system can determine locations of the surface anatomical structures of the patient based on the locations of other recognized surface anatomical structures on one or more other patients. The computing system can use machine learning or artificial intelligence algorithms to identify the patient as being a human body by detecting a silhouette of the patient, recognizing body parts of the detected silhouette (e.g., limbs, crotch, armpits, or neck), and then determine the location of additional surface anatomical structures based on the recognized body parts and known spatial relationships between surface anatomical structures stored on the memory.

Surface anatomical structures of the patient is defined as anatomical structures that can be seen with the human eye without the aid of medical imaging devices. These surface anatomical structures have known spatial relationships with each other and with underlying organs and structures. For example, the left nipple is located at the fourth rib above the heart. The umbilicus (belly button) is located at or just proximal to the bifurcation of the abdominal aorta into the two common iliac arteries and lies at the vertebral level between the 3^{rd} and 4^{th} lumbar vertebrae of the back. These known spatial relationships are saved on the memory or other database for the computing system to reference. Examples of surface anatomical structures can include, but are not limited to, the following: ears; a mastoid process; a mandible including mental region; a sternocleidomastoid muscle; an external jugular vein; anterior and posterior triangles of the neck and their smaller subdivisions; thyroid cartilage of the neck; cricoid cartilage; sternal notch and sternal angle of the sternum; xypho-sternal angles of the sternum; a clavicle and its midpoint; an acromioclavicular joint; ribs; nipples; an umbilicus; a scapula; a spinous processes of vertebrae including vertebra prominens; an axilla; a median and lateral epicondyles of the upper extremities; tuberosity of a scaphoid bone of a hand; landmarks of a femoral triangle of a thigh; a epicondyles of a femur and tibia; a greater trochanter; a patella of legs; a tibial tuberosity; iliac prominences of the pelvic girdle; a symphysis pubis; or the like.

As mentioned above, the computing system can assign surface anatomical structures based on a user input, such as touch, voice, a hand gesture, or the like. For example, if the computing system is unable to locate surface anatomical structures due to the weight, skin pigmentation, or other uncommon features of the patient, the user can input at least one or more surface anatomical structures. The computing system can then determine other surface anatomical structures based on the at least one or more entered surface anatomical structure by performing the steps described above. For example, in certain embodiments the user touches the screen at locations corresponding to a surface anatomical structure. In other embodiments, the computing system receives an input from the user via the camera, a microphone, a keyboard, a selection from a drop-down menu, or other user interface.

The computing system can determine an anatomical profile of the patient. The anatomical profile can include a plurality of characteristics corresponding to the individual. In certain embodiments, the anatomical profile includes or is based on a plurality of target data, such as age or sex of the patient. In certain embodiments, the computing system determines the anatomical profile based on an input such as touch, hand gesture, or the like from the user. In certain embodiments, the computing system uses machine learning or artificial intelligence algorithms to determine the anatomical profile. For example, the computing system determines the anatomical profile based on a plurality of target data determined by the computing system.

Once the computing system locates the necessary surface anatomical structures, the computing system can calculate and catalogue the known spatial relationships between the surface anatomical structure and corresponding internal anatomical structures. In certain embodiments, the computer system can determine the distance between each of the external anatomical structures to increase accuracy for locating and determining a size of the target internal anatomical structures. The computing system can perform a look up in the memory or other database with data that provides known spatial relationships between the surface anatomical structures and the corresponding internal anatomical structures.

The computing system then determines a probe location to place the ultrasound probe on the patient based on the processed image data and the user input. The probe location is on a surface of the patient in which the ultrasound probe, when placed against the surface at the probe location, is capable of imaging the target internal anatomical structure of the patient. The computing system determines the probe location by locating the target internal anatomical structure of the patient based on the known spatial relationships between the surface anatomical structures and the corresponding internal anatomical structures. The computing system then determines where on the patient the ultrasound probe can be placed to obtain ultrasound images of the target internal anatomical structure. Known relationships between internal anatomical structures and locations on the surface of the patient where ultrasound probes can be placed to image the corresponding internal anatomical structures can be saved on the memory of the computing system for reference.

The computing system then generates visual based augmented reality output data that is presented on the display of the augmented reality device. The visual based augmented reality output data is a digital content that includes at least one target indicia. The target indicia overlays an image of the patient on the display. The image of the patient is the actual physical patient seen directly by the user's eye(s) or image data representing the actual physical patient. The target indicia is overlayed on the display at the probe location in which the user is to place the ultrasound probe on the patient to obtain ultrasound images of the target internal anatomical structure.

The computing system can determine a proximity of the augmented reality device and the probe location on the patient via the camera, depth sensors, or other sensors. If a smart phone or a tablet is used as the augmented reality device, a live feed of the patient can be shown on the display, representing a real-world image of the actual physical patient. The computing system recognizes the body of the patient and overlays the target indicia on the live feed of the patient, providing an estimated probe location to place the ultrasound probe. If a head mount, such as smart glasses, is used as the augmented reality device, the at least partially transparent display allows the user to see the patient therethrough. The computing system detects that the at least partially transparent display is facing the patient. The at least partially transparent display shows the target indicia overlayed on the patient seen through the at least partially transparent display. The user is now guided to place the ultrasound probe against the patient at the target indicia. If the user selected an ultrasound imaging procedure, the computing system can generate a plurality of target indicias that are displayed simultaneously or in a sequential order depending on the selected procedure.

Alternatively, the computing device can use the camera to take a still picture or record a video of the patient. The computing system can then overlay the target indicia for the target internal anatomical features onto the still picture or recorded video of patient. The computing system can then display the still picture or recorded video with the overlaid target indicia on the display and the user can use the still picture or recorded video as a guide for placement of the ultrasound probe on the patient.

The target indicia can be an X, an arrow, a circle, a cross hairs, or other indicator that can precisely indicate the probe location of the ultrasound target. In certain embodiments, the target indicia can be a depiction of the internal anatomical structure of the ultrasound target. For example, if the target is the heart of the patient, the target indicia can be an image of a heart overlayed at the probe location where the heart of the patient can be imaged. In certain embodiments, the computing system only shows the target internal organ or structure to be imaged and does not show other internal organs or structures so that the user knows exactly where to place the ultrasound probe. In other embodiments, the computing system can show all of the internal organs or structures of the patient, highlight the target organ or structure, and/or dim a remainder of the non-target organs or structures.

In certain embodiments, the computing system can further aid the user to place the probe over the correct probe location of the patient by providing visual or oral cues. For example, the computing system can generate a user notification when the ultrasound probe is disposed over the probe location. The user notification can be a visual notification on the display. For example, a check mark or a flash can appear on the display indicating that the user has placed the ultrasound probe at the correct probe location of the patient. Alternatively, the user notification can be an oral notification, such as a sound projected from a speaker of the guidance system. For example, a recognizable noise can be projected from the speaker when the user has placed the ultrasound probe at the correct probe location of the patient. Thus, when the user places the ultrasound probe over the intended target, the user can be instantly notified that the user has placed the ultrasound probe at the correct probe location of the patient.

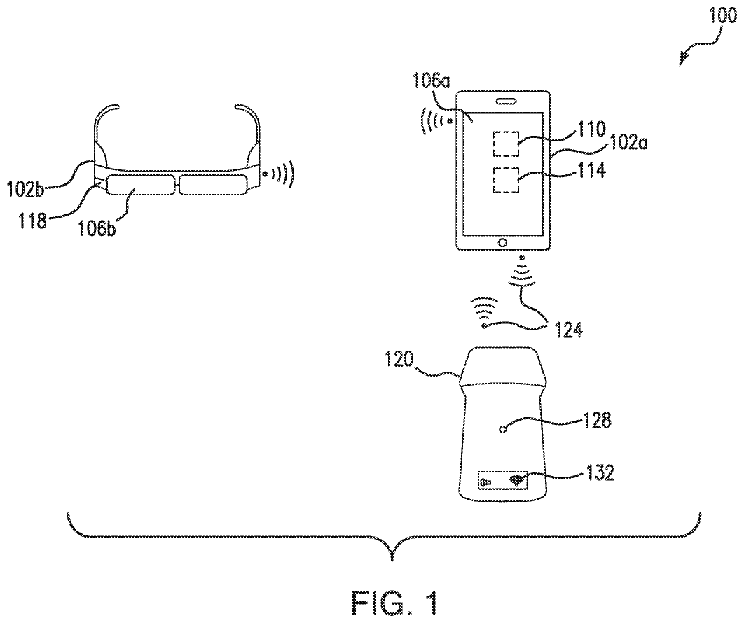

FIG. 1 depicts components of an ultrasound guidance system 100, according to an exemplary embodiment of the present invention. Ultrasound guidance system 100 can include an augmented reality device 102a, 102b. As illustrated in FIG. 1, augmented reality device 102a, 102b can be a smart phone 102a, a head mount 102b, or the like. Smart phone 102a has a touchscreen display 106a. Smart phone 102a can further include a camera (not shown). Head mount 102b has an at least partially transparent display 106b. Head mount 102b can also include a camera 118. Ultrasound guidance system 100 can further include a computing system 110, 114 that includes at least a processor 110 and a memory 114. As depicted in FIG. 1, computing system 110, 114 can be part of the smart phone 102a. Alternatively, computing system 110, 114 can be part of head mount 102b or can be separate from augmented reality device 102a, 102b, as a desktop, laptop, or the like.

Ultrasound guidance system 100 can further include an ultrasound probe 120. Ultrasound probe 120 can be configured to communicate with computing system 110, 114 over a hard wire interface or a wireless interface 124. Ultrasound probe 120 can house internal and external components. Internally, ultrasound probe 120 can include a piezoelectric ultrasound transducer and/or semiconductor chips. Ultrasound probe 120 can further include an analog to digital signal converter, a wireless transmitter, and battery. A button 128 can be on the exterior of the ultrasound encasement that allows for functional interaction with the software and device operability. Externally, ultrasound probe 120 can include a USB port or other type of charging port, and a screen 132 that displays Wi-Fi connectivity, battery level, and/or other data.

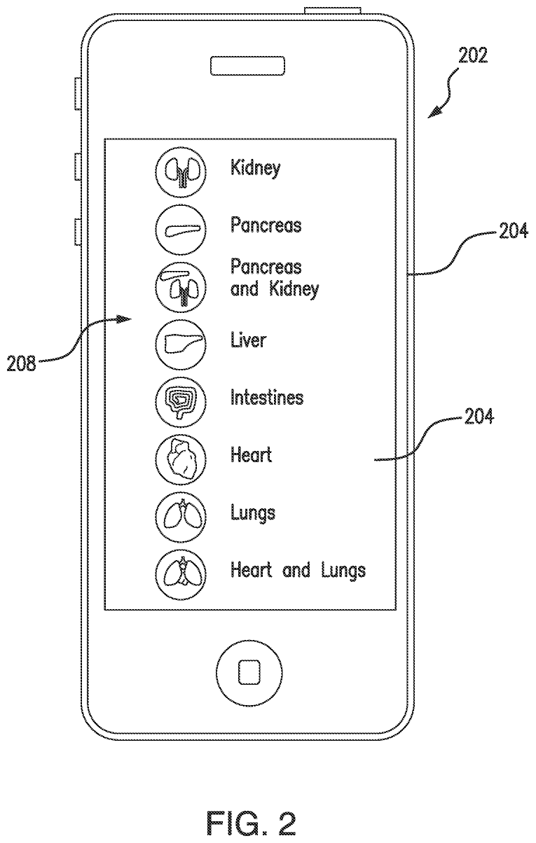

FIG. 2 depicts a computing system 202 of the present invention. Computing system 202 can be part of a smart phone 206 having a touchscreen interface 204. Touchscreen interface 204 allows for computing system 202 to receive a user input from the user. The user input includes a target internal anatomical structure of the patient that is intended to be imaged by ultrasound. The user of the present invention can start the process by selecting a target internal anatomical structure on computing system 202. Selection can be performed via touchscreen interface 204 or other interfaces described in detail above. The user can select the target internal anatomical structure from a display menu 208 that provides a plurality of target internal anatomical structures to choose from. For example, the user can select the liver, the pancreas, the kidneys, the lungs, the heart, the stomach, or the like from display menu 208. Once selected, the computing system 202 can store the user input on the memory.

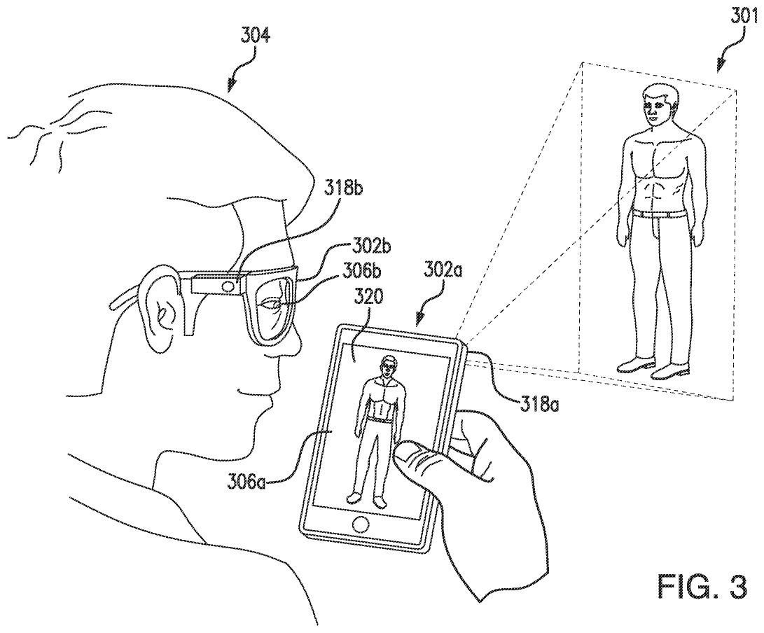

FIG. 3 depicts a user 304 pointing a camera 318a of a smart phone 302a at a patient 301 to acquire image data 320 of surface anatomy of patient 301. Alternatively, image data 320 can be acquired by a camera 318b of a head mount 302b. Image data 320 acquired by camera 318b of head mount 302b can be displayed on a display 306b of head mount 302b, display 306a of smart phone 302, or both. Image data 320 of patient 301 can be acquired before or after user 304 inputs the target internal anatomical structure of patient 301. Image data 320 can be in the form of a still image, a video, or a live feed of patient 301. For example, user 304 can prompt the computing system to capture an image or video of patient 301 via camera 318a, 318b. Alternatively, user 304 can simply point camera 318a, 318b in the patient's direction and the computing system can recognize a live feed of patient 301.

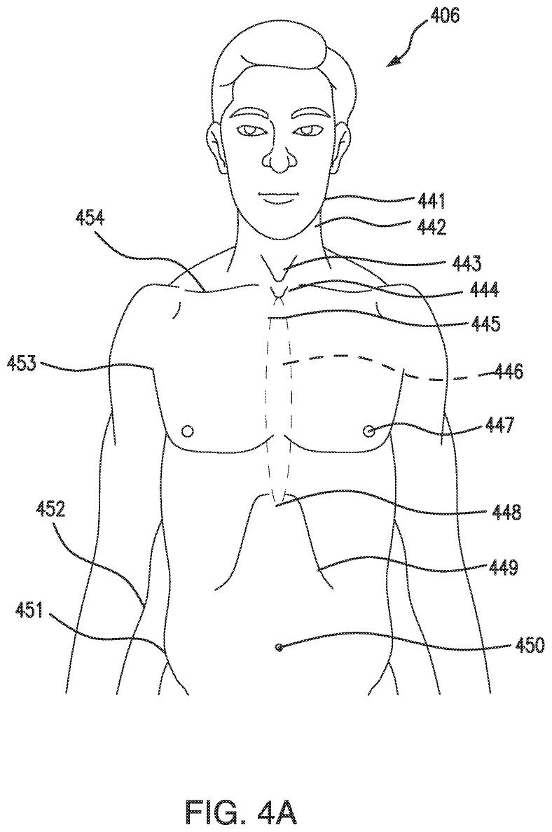

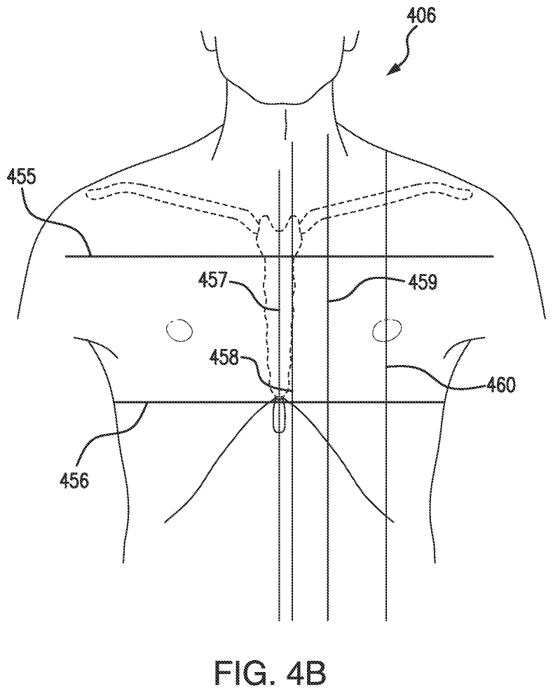

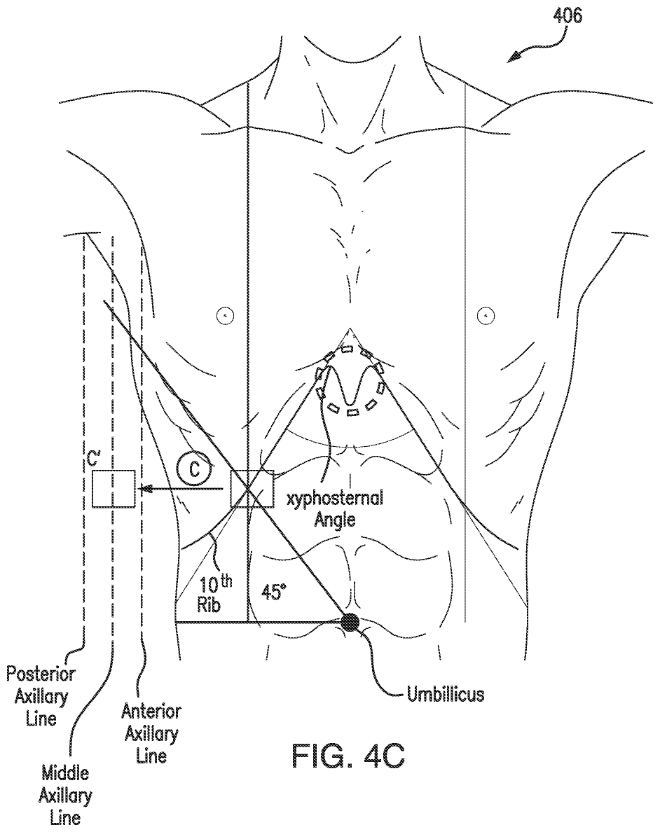

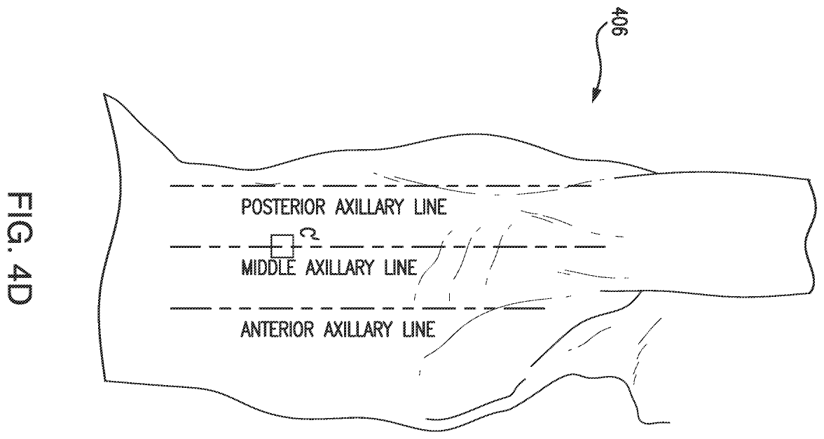

FIGS. 4A-4D show images of surface anatomy of patient 406 to be processed by the computing system. The computing system processes the image data by identifying surface anatomical structures of the patient. In certain embodiments, the computing system recognizes one or more particular surface anatomical structures of the patient, as described in detail above. Examples of surface anatomical structures that can be identified by the computing system are shown in FIG. 4A and can include: mandible 441; neck 442; thyroid cartilage 443; sternal notch 444; sternal angle 445; sternum 446; nipple 447; xyphoid process of sternum 448; ribs 449; umbilicus 450; iliac crest of pelvis 451; median epicondyle 452; axilla 453; and clavicle 454. The above list should not be considered limiting as the computing system can recognize other surface anatomical structures in an image of a patient, such as surface anatomical structures on the patient's back, legs, arms, head, and face.

The computing system is capable of using guide lines in combination with other recognized surface anatomical features that further aid the computing system in identifying locations of other surface anatomical structures and underlying internal anatomical structures. The surface anatomical structures have known spatial relationships between one another and with the underlying internal anatomical structures that are saved in the memory. Once a surface anatomical structure is identified, the computing system can use the guide lines that provide guides for the known spatial relationships between the surface anatomical structures. Thus, the computing system identifies a first surface anatomical structure and makes calculations of other surface anatomical structures to determine the known locations of the underlying internal anatomical structures. Examples of guide lines that can be used are illustrated in FIG. 4B and can include the following: sternal angle 455; xipho-sternal line 456; midline 457; lateral sternal line 458; the parasternal line 459; and the midclavicular line 460. Other guide lines can be used when determining locations of surface anatomical structures and underlying internal anatomical structures.

FIGS. 4C and 4D depict an exemplary method of a computing system determining locations of surface anatomical structures and an underlying internal anatomical structure using multiple images of patient 406. As an example, if a user selects the focused abdominal sonography for trauma (FAST) ultrasound exam, the computing system can first determine a location of a 10^{th} rib and a midaxillary guide line. The 10^{th} rib is located at the intersection of guidelines generated using the umbilicus, midclavicle and xyphosternal angle. The midaxillary guide line is a line drawn straight down from a middle of the armpit on a side of patient, as best shown in FIG. 4D. The location of the 10^{th} rib is C. The location C is moved laterally to the midaxillary line to location C′. The location C′ is a target region where an ultrasound probe can image an interface of the lung, liver, and kidney and is a first location that is imaged for the FAST ultrasound exam. The target indicia can overlay the image of patient 406 at C′ so that the user can place the ultrasound probe at the location of C′.

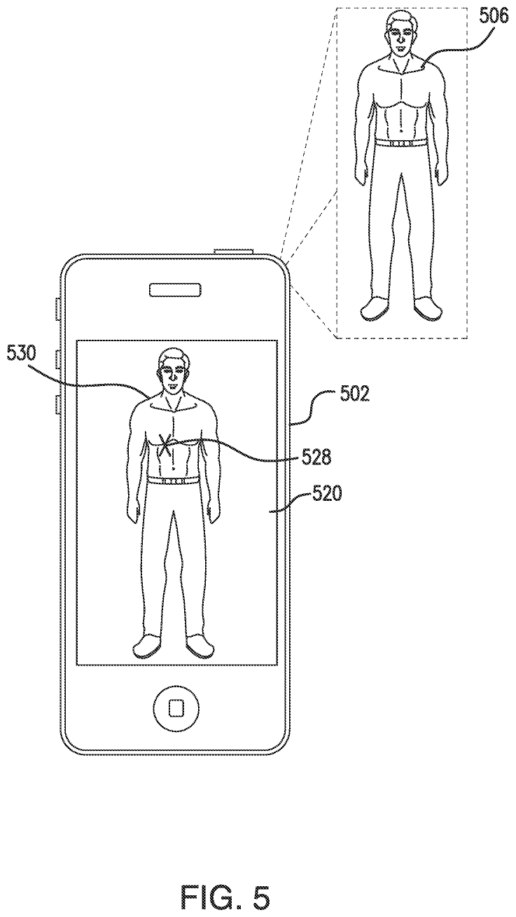

FIG. 5 depicts target indicia 528 overlayed on an image 530 of a patient 506. As mentioned above, the computing system generates output data for display 520 of augmented reality device 502. The output data is target indicia 528 that overlays image 530 of patient 506 on display 520 at the probe location where the ultrasound probe is capable of imaging the target internal anatomical structure. The probe location is on a surface of the patient where the internal anatomical structure is underneath and/or can be imaged from. The computing system can determine a proximity of the augmented reality device 502 relative to patient 506 via the camera or other sensors. As illustrated in FIG. 5, augmented reality device 502 is a smart phone and display 520 is a touchscreen. In such embodiments, a live feed of patient 506 can be shown on display 502, representing a real-world image. The computing system recognizes the body of patient 506 and overlays target indicia 528 on the live feed of patient 506 at the probe location, providing an estimated location to place an ultrasound probe.

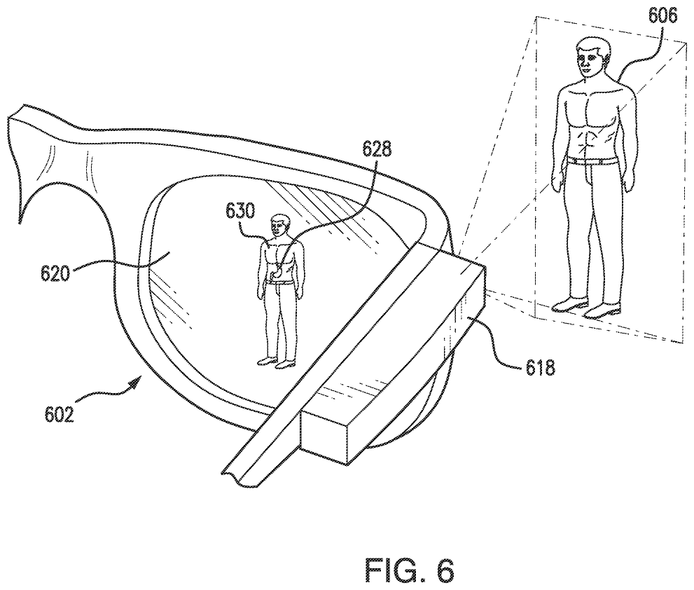

FIG. 6 depicts target indicia 628 overlayed on an image 630 of a patient 606. In this embodiment, an augmented reality device 602 is a head mount, such as smart glasses. Display 620 of augmented reality device 602 is an at least partially transparent display 620 that allows the user to see patient 606 therethrough. The computing system detects that display 620 is facing patient 606 by a camera 618 or other sensors. At least partially transparent display 620 shows target indicia 628 overlayed on image 630 of patient 606 seen through display 620. The user is now guided to place the ultrasound probe against patient 606 at target indicia 628. FIG. 6 shows an example in which the target indicia is a depiction of a target organ of patient 606.

The disclosure herein refers to certain illustrated examples, it is to be understood that these examples are presented by way of example and not by way of limitation. The term “about,” as it appears herein, is intended to indicate that the values indicated can vary by plus or minus 5%. The intent of the foregoing detailed description, although discussing exemplary examples, is to be construed to cover all modifications, alternatives, and equivalents of the examples as can fall within the spirit and scope of the invention as defined by the additional disclosure.

The entire contents of all cited references in this disclosure, to the extent that they are not inconsistent with the present disclosure, are incorporated herein by reference.

The present invention can include any combination of the various features or embodiments described above and/or in the claims below as set forth in sentences and/or paragraphs. Any combination of disclosed features herein is considered part of the present invention and no limitation is intended with respect to combinable features.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

What is claimed is:

1. A guidance system comprising:
an augmented reality device having a display;
a camera configured to capture image data; and
a computing system comprising a processor and a memory, wherein the memory has stored therein computer-readable instructions that, upon execution by the processor, configure the computing system to
receive a user input from a user, the user input comprising a target internal anatomical structure of the patient,
process the image data captured by the camera, the image data comprising at least an image of surface anatomy of the patient, wherein processing the image data comprises identifying at least one surface anatomical structure of the patient to locate internal anatomy of the patient,
determine a probe location, based on the processed image data and the user input, wherein the probe location is on a surface of the patient where an ultrasound probe, when placed against the surface at the location, is capable of imaging the target internal anatomical structure of the patient, and
generate output data for the display of the augmented reality device, the output data comprising a target indicia, wherein the target indicia overlays an image of the patient, on the display, at the probe location.

2. The guidance system of claim 1, further comprising an ultrasound probe configured to generate ultrasound images, wherein the ultrasound probe is communicatively coupled to the computing system.

3. The guidance system of claim 2, wherein the computing system generates a user notification when the ultrasound probe is disposed at the probe location.

4. The guidance system of claim 3, wherein the user notification is a visual notification on the display.

5. The guidance system of claim 3, further comprising a speaker, wherein the user notification is a sound projected from the speaker.

6. The guidance system of claim 1, wherein the augmented reality device comprises the camera.

7. The guidance system of claim 1, wherein the augmented reality device is a smart device.

8. The guidance system of claim 1, wherein the augmented reality device comprises a head mount and the camera, wherein the display is an at least partially transparent display.

9. The guidance system of claim 8, wherein the computing system determines a proximity measurement between the head mount and the probe location on the patient, and the target indicia overlays the probe location on the patient on the at least partially transparent display when the computing system determines that the at least partially transparent display is facing the patient.

10. The guidance system of claim 1, wherein the target indicia is a depiction of the target internal anatomical structure.

11. A method of guiding a user to capture ultrasound images, comprising:
entering a user input to a computing system, the user input comprising a target internal anatomical structure of a patient;
capturing, with a camera, image data of surface anatomy of the patient;
processing, with the computing system, the image data captured by the camera, wherein processing the image data comprises identifying at least one surface anatomical structure of the patient, to locate internal anatomy of the patient;
determining, with the computing system, a probe location based on the processed image data and the user input, on a surface of the patient where an ultrasound probe, when placed against the surface at the probe location, is capable of imaging the target internal anatomical structure of the patient; and
generating, with the computing system, output data for a display of an augmented reality device, the output data comprising a target indicia, wherein the target indicia overlays an image of the patient on the display at the probe location.

12. The method of claim 11, further comprising
placing an ultrasound probe against the patient's body at the probe location.

13. The method of claim 12, further comprising generating, with the computing system, a user notification when an ultrasound probe is disposed at the probe location.

14. The method of claim 13, wherein the user notification is a visual notification on the display.

15. The method of claim 13, wherein the user notification is a sound projected from a speaker.

16. The method of claim 11, wherein the augmented reality device comprises the camera.

17. The method of claim 11, wherein the augmented reality device is a smart device.

18. The method of claim 11, wherein the augmented reality device comprises a head mount and the camera, wherein the display is an at least partially transparent display.

19. The method of claim 18, further comprising
determining, with the computing system, a proximity measurement between the head mount and the probe location of the patient, and
overlaying, with the computing system, on the at least partially transparent display, the target indicia over the probe location on the patient, such that the target indicia is viewed by the user through the at least partially transparent display when the computing system determines that the at least partially transparent display is facing the patient.

20. The method of claim 11, wherein the target indicia is a depiction of the target internal anatomical structure.
